(19) United States
(12) Patent Application Publication
(10) Pub. No.: US 2022/0246289 A1
(43) Pub. Date: Aug. 4, 2022

(54) Modular Digital Treatment System

(71) Applicant: Closed Loop Medicine Ltd., Cambridge (GB)

(72) Inventors: David Tonatiuh Cox, London (GB); James Matthew Siddle, London (GB); Jakub Kammer, Brentford (GB)

(73) Assignee: Closed Loop Medicine Ltd., Cambridge (GB)

(21) Appl. No.: 17/591,348

(22) Filed: Feb. 2, 2022

Related U.S. Application Data
(60) Provisional application No. 63/145,077, filed on Feb. 3, 2021.

(51) Int. Cl. G16H 40/40; G16H 20/00; G06F 8/71

(52) U.S. Cl. CPC G16H 40/40; G16H 70/40; G06F 8/71; G16H 20/00

(57) ABSTRACT

A digital treatment system, comprising: a core package; and a treatment package; wherein the core package is software partitioned from the treatment package, wherein the core package is adapted to: receive a treatment configuration from the treatment package; and configure one or more patient treatment therapies based on the treatment configuration.

### CROSS-REFERENCE TO RELATED APPLICATIONS

This patent application claims priority to, and incorporates by reference the entire disclosure of, U.S. Provisional Patent Application No. 63/145,077, filed on Feb. 3, 2021.

### BACKGROUND

The present disclosure relates to methods and systems for providing digital therapy, in particular to modular digital treatment systems and operation thereof.

Patients may receive treatment for one or more diseases or conditions via a Digital Therapeutic (DTx). A DTx comprises software which itself can convey therapeutic benefit to a patient without the involvement of a human healthcare professional. Therefore, a DTx is effectively the use of “software as medicine”. As a therapeutic, DTx can require regulatory approval from pharmaceutical regulators. As software, DTx also falls under the definition of a Medical Device, as defined by the FDA (200 CFR 80) and EU & UK (Regulation (EU) 2017/745).

Regulators are primarily concerned with 3 aspects of “Software as a Medical Device” (SaMD):
Intended Use
Efficacy
Safety

Intended Use is often defined by the manufacturer of the SaMD. The efficacy and safety are typically proven through clinical trials, which are expensive and take a long time to conduct.

The modern software development paradigm is to release software as early in its development as possible, and then iterate rapidly with incremental improvements, taking into account usage and feedback by the end-user in the real world.

This is at odds with SaMD, since, given the burden associated with attaining regulatory approval (cost and time), a manufacturer would not want to iterate the SaMD once it had entered a clinical trial. However, this may preclude the ability to observe the SaMD in use in the real world and improve it accordingly.

The regulatory burden may also prevent the SaMD from incorporating the latest knowledge, research or guidelines for a given treatment or therapy in order to avoid amendment of the SaMD package. This problem may be exacerbated for a DTx or SaMD comprising co-therapies (multiple therapies) for a respective disease or condition. For example, the DTx may comprise a dosage regimen for a pharmacological therapy in combination with a non-pharmacological therapy, such as cognitive behavioural therapy (CBT) or an exercise regimen. The problem may yet be further exacerbated for a SaMD based digital treatment system configured to treat multiple diseases or conditions.

In view of the above, there is a need for systems and methods for providing digital therapy that can be updated and improved, based on new developments relating to use of the SaMD and changes in knowledge and guidance relating to one or more particular therapies, in a way that is compliant with regulatory requirements without being unduly burdensome.

### SUMMARY

According to a first aspect of the present disclosure there is provided a digital treatment system, comprising:
a core package;
a treatment package; and
an interface arranged to software partition the core package from the treatment package,
wherein the core package is adapted to:
   receive a treatment configuration from the treatment package; and,
   configure one or more patient treatment therapies based on the treatment configuration.

By software partitioning the core package from the treatment package, the two parts can be independently assessed for regulatory compliance. As a result, the two parts may also be updated independently where the impact on the other is within acceptable bounds according to the regulations. Separating the digital treatment system into two (or more) parts allows the separation of software components or modules dependent upon their regulatory burden and/or expected frequency of iteration.

The system can be partitioned to provide general purpose components in the core package and treatment specific components in the treatment package. In this way, the core package can be used with multiple treatment packages together or independently without requiring reapproval of the core package for each treatment.

The disclosed digital treatment systems may provide one or more of the following benefits:

For the user:
The system can provide a consistent user interface experience, whilst supporting different treatments
The system may run several different treatments simultaneously
The system can manage and rationalise requests for action arising from multiple treatments (for example, two treatments may both require a blood pressure reading. The system can rationalise the data requirements, issue a single notification to the patient and relay the resulting measurement result to multiple treatments.
The system may provide continuity of user personalisation from one treatment to the next

For the system provider:
Segregation of functionality can ease regulatory burden for new/updated treatments or functionality
Enables easier/more frequent iterations of components not subject to high regulatory scrutiny

The digital treatment system may be a SaMD and may provide one or more digital therapies.

While the disclosure is amenable to various modifications and alternative forms, specifics thereof have been shown by way of example in the drawings and are described in detail. It should be understood, however, that other implementations, beyond the particular implementations described, are possible as well. All modifications, equivalents, and alternative implementations falling within the spirit and scope of the appended claims are covered as well.

The above discussion is not intended to represent every example implementation or every implementation within the scope of the current or future Claim sets. The figures and Detailed Description that follow also exemplify various example implementations. Various example implementations may be more completely understood in consideration of the following Detailed Description in connection with the accompanying Drawings.

According to a second aspect of the present disclosure there is provided a digital treatment system, comprising:
a treatment package relating to a patient therapy;
a core package comprising a core platform, the core package adapted to configure the patient therapy; and
a plurality of software modules including:
   one or more core modules in the core package; and/or
   one or more treatment modules in the treatment package,
wherein the core platform is configured to integrate the plurality of software modules such that the core package is software partitioned from the treatment package.

The core platform may (safely) integrate the plurality of software modules by implementing an instrument registration and matching process as described below in relation to FIG. 4. The core platform may control communication between the plurality of software modules to provide the software partition.

In one or more examples, there may be provided a computer program, which when run on a computer, causes the computer to configure any apparatus, including a circuit, controller, converter, or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software may be an assembly program.

The computer program may be provided on a computer readable medium, which may be a physical computer readable medium such as a disc or a memory device, or may be embodied as a transient signal. Such a transient signal may be a network download, including an internet download. There may be provided one or more non-transitory computer-readable storage media storing computer-executable instructions that, when executed by a computing system, causes the computing system to perform any method disclosed herein.

### BRIEF DESCRIPTION OF THE DRAWINGS

One or more implementations will now be described by way of example only with reference to the accompanying drawings in which:

FIG. 1 illustrates an example digital treatment system according to an implementation of the present disclosure;

FIG. 2 illustrates another example digital treatment system according to an implementation of the present disclosure;

FIG. 3 illustrates a further example digital treatment system according to an implementation of the present disclosure;

FIG. 4 illustrates an example certification and contract specification process for use in a digital treatment system according to an implementation of the present disclosure; and

FIG. 5 illustrates an example end-to-end process for user interaction with the digital treatment system according to an implementation of the present disclosure.

### DETAILED DESCRIPTION

The digital treatment system according to the first aspect defines a core package software partitioned from one or more treatment packages for configuring patient treatment therapies. By software partitioning the core package from the treatment package, the two parts can be independently assessed for regulatory compliance. Separating the digital treatment system into two or more parts allows the separation of software components or modules dependent upon their regulatory burden and/or expected frequency of iteration.

Software partitioning the digital treatment system can comprise locating components in the core package or treatment package dependent upon an expected level of regulatory scrutiny and/or frequency of update. For example, machine learning algorithms can be the subject of rigorous regulatory scrutiny and can incur a high cost and approval delay if updated on a regular basis (outside the bounds of any pre-agreed model improvements).

The core package can be used with a plurality of treatment packages. As a result, the core package can provide common capabilities for a range of treatments advantageously avoiding re-evaluation of the core package as new treatments are developed.

In one example, the treatment package may contain high-regulatory burden components. As described herein, a high-regulatory burden component may be one which contains particularly high-risk medical device software and requires scrutiny by the regulator. For example, a high-regulatory burden component may require one or more clinical trials and/or may not be updated without being subject to one or more further clinical trials. The treatment package can be stable and thereby incur the cost of the high burden as few times as possible, ideally only once. The core package can then contain components that do not attract high regulatory scrutiny, for example non-medical components. Advantageously, some non-medical components can be iterated rapidly to the benefit of the combination (and ultimately the patient) while maintaining the Intended Use, expected Efficacy, or proven Safety of the DTx within acceptable bounds.

In another example, the core package may contain high-regulatory burden components and undergo regulatory approval only once or a few times. The core package can then advantageously interface with one or more new treatment packages and improved treatment packages as they are developed without being subject to further clinical trials. The new treatment packages may be the subject of their own independent regulatory assessment. In some examples, the treatment packages may also comprise high-regulatory burden components. However, the treatment packages can be assessed independently without requiring full reassessment of the core package or other approved treatment packages.

In yet further examples, the core package and/or the treatment package may themselves be further subdivided into modules with differing requirements of regulatory scrutiny and update frequency.

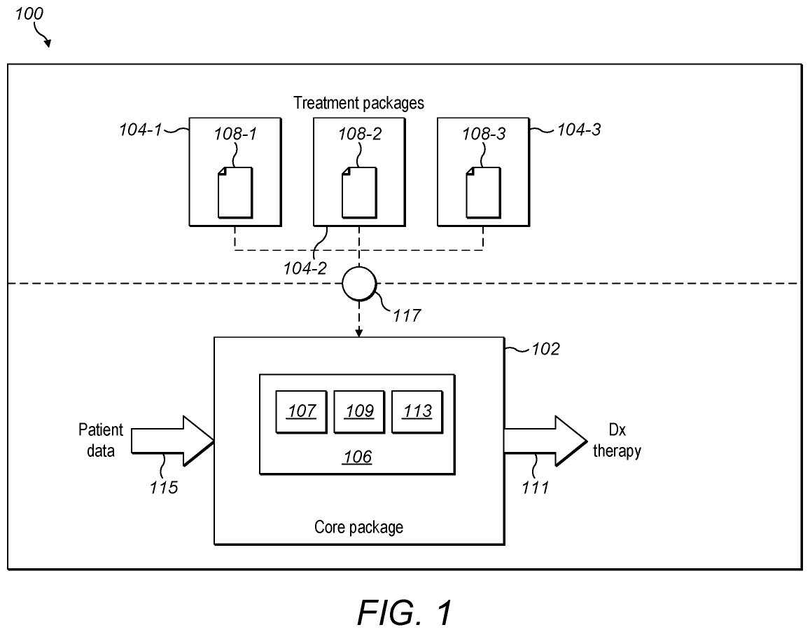

FIG. 1 shows a schematic of an example digital treatment system 100 according to an implementation of the present disclosure.

The digital treatment system 100 comprises a core package 102 and a plurality of individual treatment packages 104-1, 104-2, 104-3, collectively referred to as treatment packages 104. The core package 102 is software partitioned (or separated) from the plurality of treatment packages 104. In other words, the core package 102 and the treatment packages 104 are separate, distinguishable packages, or blocks of code, that are contained independently within the digital treatment system 100. In this example, the core package 102 comprises a treatment configurator 106. Each treatment package 104-1, 104-2, 104-3 comprises a respective treatment configuration 108-1, 108-2, 108-3. The treatment configurator 106 can receive a treatment configuration 108 from a respective treatment package 104 and configure one or more patient therapies 111 based on the treatment configuration 108.

The digital treatment system 100 is subdivided into two parts—the core package 102 and one or more treatment packages 104. The two parts can communicate via an interface 117. The core package 102 can provide a set of common capabilities to the one or more treatment packages 104. In this way, the core package 102 can enable different digital therapeutics to meet their intended use. The digital treatment system 100 may be considered as a medical device and may deliver safety-critical treatments.

By separating the treatment system 100 into two parts—the core package 102 and the one or more treatment packages 104—the two parts can be subject to independent regulatory assessment. The two parts may also be updated independently.

The concept of the core package 102 and separate treatment packages 104 can be metaphorically analogised as a portable games console with the core package 102 representing the handheld console and the treatment packages 104 representing individual game cartridges.

A treatment package 104 may be considered as a delivery mechanism used to make therapeutic software, application configuration, and related assets available to the core package 102 and system 100 so that a specific treatment can be made available to a user.

A Treatment package 104 may include:
A definition of specific treatment modules required for the treatment
The executable code of the specific treatment modules required to deliver the treatment.
A treatment descriptor defining the clinical workflow and decisions to be made as part of the treatment.
An application configuration used to configure modules of the core package 102 or other parts of the system 100.

Further detail on the components of the treatment package 104 is provided below.

The core package 102 can provide shared capabilities to the treatment packages 104. These shared capabilities can include data management, prescription checking and third-party device integration. The capabilities may further include capabilities more closely involved in treatment delivery such as the execution of the treatment configuration 108 by the treatment configurator 106, described in more detail below.

The core package can configure the one or more digital treatment therapies 111 by performing a number of operations associated with the treatment (as indicated by the treatment configuration). For example, the core package may: acquire data; process treatment decisions; execute algorithms; notify the patient or health care provider; perform user authentication; and monitor compliance as part of configuring the one or more digital treatment therapies 111.

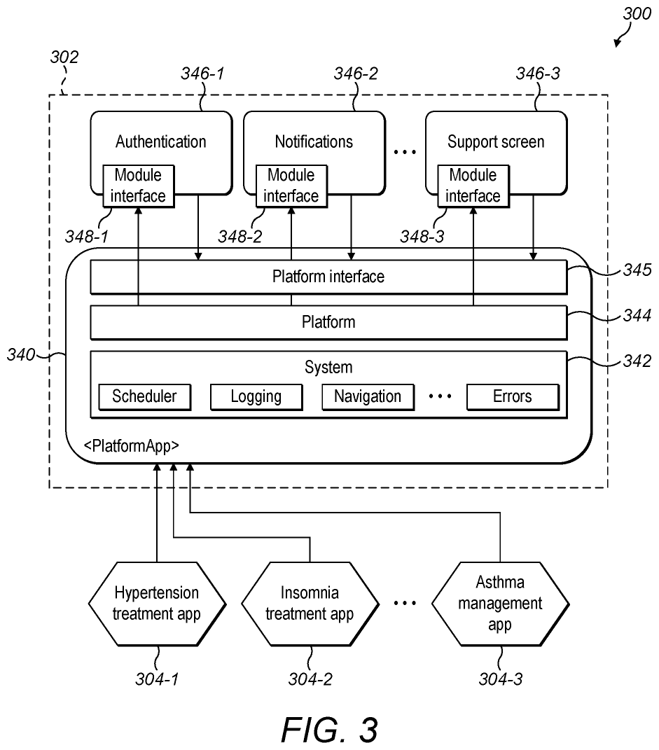

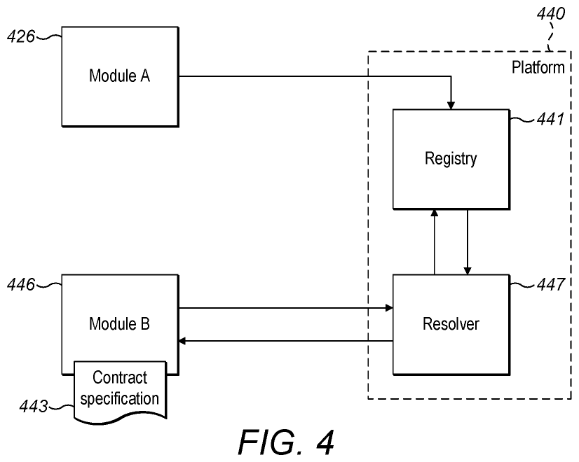

In some examples, the core package 102 may comprise a core platform with a plurality of core modules operating on the core platform. The core platform may comprise a platform application or application framework providing a base system layer upon which functionality can be provided by the core modules. Each core module may be a separated partition of software with one or more performance properties. These performance properties may comprise any of: module functionality provided by the module; module performance constraints required by the module of the digital treatment system (or other modules) in order to operate correctly and safely; inbound information constraints and outbound information constraints on data or information being passed to or from the module; and descriptive properties for enabling one or more other modules and/or the digital treatment system to determine compatibility of the module with performance constraints of the respective other module or digital treatment system as a whole. These performance properties are discussed further below in relation to FIGS. 3 and 4.

Core modules can be common modules that provide functionality to more than one treatment package.

The core modules may comprise medical core modules that provide medical-device functions for delivering the digital treatment. For example, a notification module may be considered as a medical core module as notifying or instructing the user to perform an action, such as take a drug or perform a therapeutic task, may comprise part of the treatment.

The core modules may comprise non-medical core modules that provide non-medical-device functions. For example, an authentication module may be considered as a non-medical core module because authenticating a user account is not related to the delivery of the actual therapy.

The core modules may be considered as common modules as they can be incorporated into many treatments via access from a corresponding treatment package. In some examples, the core modules may comprise IEC 62304-compliant software units that can be “taken off the shelf” and included in the medical device system.

Each treatment package 104 may also comprise one or more modules in the form of treatment specific treatment modules. In the same way as the core modules, the treatment modules may also comprise separated partition of software with one or more performance properties. The core package 102 may execute the treatment modules on the core platform.

Implementing the digital treatment system in a modular format enables the independent updating and/or regulatory approval of the system parts. That is, the platform, each core module and each treatment package/treatment module may be updated and/or approved independently. In this way, the digital treatment system 100 can be partitioned dependent upon regulatory burden. Table 1 illustrates an example partitioning of the system 100 indicating whether each partition is medical or non-medical, a frequency of update and an interface stability.

TABLE 1

| | Med/non- | Frequency | Interface | |
|---|---|---|---|---|
| Partition | med function | of Change | Stability | Rationale |
| Platform Application | Non-medical | Frequent | Stable | Expect frequent change as new |
| | | | | shared capabilities are added |
| | | | | and underlying dependencies |
| | | | | are updated. The platform |
| | | | | interface should remain stable |
| | | | | except for non-disruptive |
| | | | | additions. |
| Core Modules (with | Non-medical | Regular | Unstable | Non-medical modules can be |
| non-medical-device | | | | updated to roll out |
| functions) | | | | improvements as they are |
| | | | | added. This may be to support |
| | | | | new treatments (for example). |
| Core Modules (with | Medical | Infrequent | Stable | Common medical device |
| medical-device | | | | components should change |
| functions) | | | | infrequently due to increased |
| | | | | risk associated with change, and |
| | | | | the resulting regulatory burden. |
| Treatment Package (inc | Medical | Infrequent | Stable | The treatment package should |
| custom, | | | | change infrequently due to |
| treatment-specific | | | | increased risk associated with |
| modules) | | | | change, and the resulting |
| | | | | regulatory burden. |

Modular Architecture

FIG. 3 illustrates an example digital treatment system according to an implementation of the present disclosure. Features of FIG. 3 which are also present in FIG. 1 have been given corresponding numbers in the 300 series and will not necessarily be described again here.

The digital treatment system 300 comprises a core package 302 and a plurality of treatment packages 304, each relating to a different disease/condition.

In this example, the core package 302 comprises a core platform in the form of a core application platform 340. The core platform 340 comprises a system layer 342, a platform layer 344 and a platform interface 345. The core package 302 further comprises a plurality of core modules 346.

The core modules 346 (and any treatment modules within the treatment packages 304) encapsulate a particular functionality of the system 300. Each module (or software unit) may be considered as an independent or isolated piece of software code having a well defined boundary. As discussed below, the modules can have one or more performance properties. In this way, the modules can have an associated risk profile and be considered as atomic for the purpose of medical device classification.

The platform interface 345 provides each module with a way to access functionality of the platform layer 344 and communicate with other modules. For many of the modules, direct communication between the modules may be inhibited and modules may only communicate with each other via the core platform 340 and/or the platform layer 344. For some modules, direct inter-module communication may only be inhibited until a module registration process as discussed below in relation to FIG. 4.

The platform layer 344 can manage communication between the modules and invoke functionality of the modules through registered module interfaces 348. The platform layer 344 may also invoke elements of the system layer 342 to provide certain core capabilities. The system layer 342 may provide functionality of the core package 302 not directly accessible to, and not directly accessing other parts of the application.

Each module may comprise a module interface 348. The module interface 348 can provide a way for the core platform 340 to interact with the respective module (eg via one or more call-backs). Each module may register the module interface 348 with the core platform 340/core package 302. In this way, each module can inform the core package 302 of its performance properties—ie the functionality provided plus any associated constraints.

The module interface 348 may comprise imported and exported instruments. An instrument may comprise a software artifact capable of executing actions or producing data related to a single, defined task. Interaction of functionality within a module with functionality outside a module may be conducted through:
Exported instruments—comprising services and data that the module can provide to other parts of the digital treatment system 300; and
Imported instruments—indicating the services and data that the module requires from other parts of the digital treatment system 300.

The platform layer 344 may assess the exported instruments of a particular module for their suitability for use in other modules, such as availability of integration or contract test results, or other risk-relevant information. The exported instruments may comprise at least some of the module performance properties. In this way, the platform layer 344 may be configured to impose one or more constraints on the interaction between modules, based on the module performance properties such as the module functionality, performance constraints and the input/output requirements.

The module interface 348 may be associated with a set of restrictions or constraints on imported instruments based on the module performance properties. In other words, each module includes an acceptable instrument policy to ensure the imported instrument meets the constraints of the module. In this way, the module interface may be configured to impose one or more constraints on information or invocations received from the rest of the system 300.

In addition, the platform may certify imported instruments as suitable for import, meaning that the imported instrument fulfils a contract specification of the importing module. The contract specification can define how imported instruments are used, and impose request/response or message-level constraints to ensure the imported instrument behaves in a way that meets the needs of the module. The contract specification may comprise or be based on the one or more module performance properties. Example constraints include “metadata x MUST be returned with each Blood Pressure measurement”, or “information about an error must be in format y”.

FIG. 4 illustrates an example module registration process in a digital treatment system according to an implementation of the present disclosure. Features of FIG. 4 which are also present in FIG. 1 or FIG. 3 have been given corresponding numbers in the 400 series and will not necessarily be described again here.

The figure illustrates the registration of instruments of a first module 426 with the core platform 440 and subsequent discovery of the instruments by a second module 446. As an example, the first module 426 may correspond to a treatment specific module associated with a treatment package and the second module 446 may correspond to a core module. The registration of the treatment specific module may occur when the corresponding treatment package is installed in the treatment system or updated. However, the following process may relate to any modules of the treatment system.

At a first step, the first module 426 may register one or more exported instruments (including the one or more performance properties) with the core platform 440. The core platform 440 may register the exported instruments in a platform registry 441. In this way, the first module can register an interface definition, properties, constraints and characteristics. The exported instruments define services and data that the module can provide to other parts of the digital treatment system. In the example of a treatment specific module, the first module may register one or more exported instruments, for example a calculated daily dose regimen related to the treatment.

At a second step, the second module 446 transmits an instrument request to the core platform 440 in an attempt to discover an instrument that meets a contract specification 443 associated with the second module 446. The second module 446 may transmit or register the contract specification to the core platform 440 as part of this process. In an example where the second module is a notification module, the module may require the calculated daily dose regimen provided by the treatment specific module 426 as an exported instrument. The core platform 440 may store or register the instrument request in a resolver 447. Although, the first two steps are described as two distinct steps, in other examples, a module may register its exported instruments based on its module performance properties (functionality offered and constraints) at the same time as discovering instruments registered by other modules also based on its required performance properties (contract specification).

In a third step, the resolver 447 may communicate with the registry 441 to compare the provided instruments of the first module 426 (or any other module that has registered instruments) with the instrument requests of the second module 446. In a fourth step, the resolver may check the provided instruments of the first module 426 against the contract specification 443 of the second module 446. If the resolver determines that the instruments of the first module 426 meet the contract specification 443 of the second module 446, the resolver may provide the instrument details to the second module 446.

The resolver 447 may provide the instrument details of the first module 426 to the second module 446 in the form of reference information to enable communication. In this way, the resolver provides the second module 446 with a reference interface to communicate with the first module 426. In some examples, the reference interface may correspond to direct communication between the first module 426 and the second module 446, in-memory, in the form of a reference to an object that can be invoked. In other examples, the reference interface may correspond to an intermediary communication such as via the core platform 440.

The module registration process of FIG. 4 and the subsequent establishment of a reference interface can define the software partition between core package and treatment package of the digital treatment system.

The modular structure of the digital treatment system outlined in FIGS. 3 and 4, including the platform, the modules and associated instrument definitions, import policies, and certification, can provide and enforce the independence between modules. This in turn enables independent variation of the parts of the system 300 without requiring regulatory re-approval of the whole system 300. The elements of the system 300 may be integrated or combined to deliver the treatments, while allowing isolation between medical/non-medical functions and independent variation of these elements.

Returning to FIG. 1, the core package 102 may include a plurality of core modules including the treatment configurator 106. The core package 102 may acquire or receive patient data 115 which may be acquired via the core platform and/or via one or more of the core modules and/or treatment specific modules. The patient data 115 may comprise biometric data, behavioural data, prescription data, physiological data, such as physiological measurement data, medical record data, desired patient outcome data, patient feedback data or any other relevant patient data. The patient data 115 may be acquired via one or more of: a patient input, a reading from an associated device and patient data collected from a local or networked database. Here the term associated device may comprise any device providing data, for example regulated medical devices (for example a glucose monitoring device) or non-medical devices such as fitness trackers, smart watches and similar devices known in the art. The core package 102 may be configured to acquire other relevant non-patient data such as environmental data or regulatory data, for example drug data or drug interaction data.

The treatment configurator 106 can receive a treatment configuration 108 from a respective treatment package 104 and configure the one or more digital therapies 111 associated with the treatment package 104. The one or more digital therapies 111 may include a recommended dosage of a pharmacological therapy, such as a drug. The pharmacological therapy may comprise a drug already prescribed to the patient. In addition, or alternatively the one or more digital therapies 111 may include a non-pharmacological therapy, such as medical device instructions or cognitive behavioural therapy. The core package 102 or a treatment specific module may communicate the medical device instructions to the patient and/or an associated medical device. The non-pharmacological therapy may comprise a therapy already prescribed to the patient.

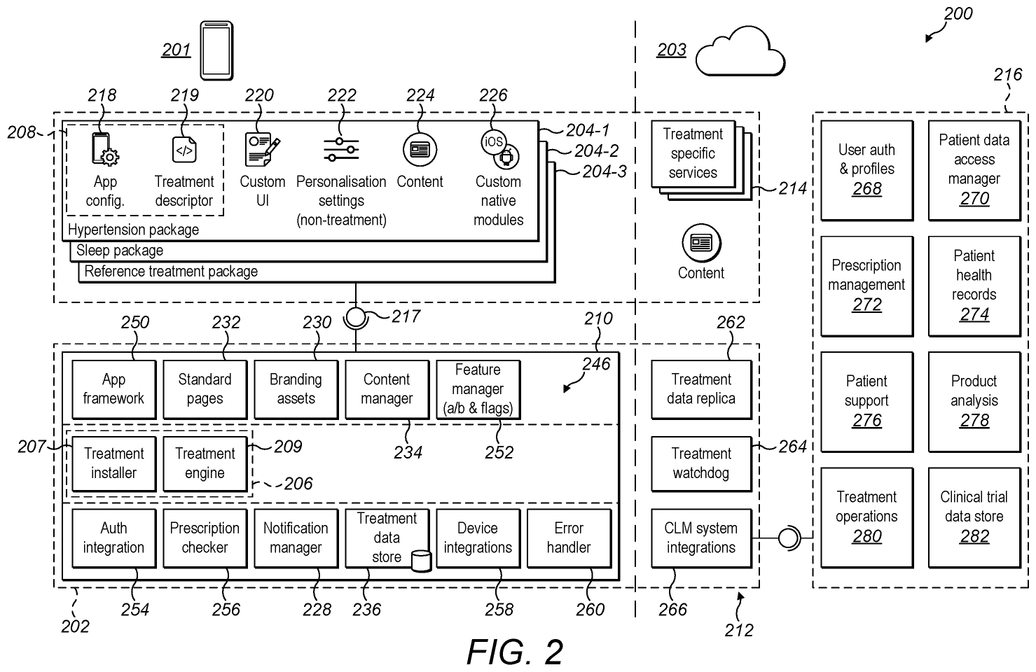

The treatment configuration 108 may include an application configuration which can indicate a list of core modules required to deliver the respective treatment. The treatment configurator 106 may receive the application configuration and register the core modules required for that treatment. The treatment configurator 106 may comprise a treatment installer 107 for installing the treatment package 104 on the core platform according to the application configuration. The application configuration is described in further detail below in relation to FIG. 2.

The treatment configuration 108 may comprise a treatment descriptor including treatment instructions in a computer readable format. The treatment instructions may include a set of treatment decisions. The treatment decisions may comprise one or more clinical (or clinically significant) decisions. The treatment configurator 106 may include a treatment engine 109 to process or execute the treatment decisions. In some examples, the treatment configurator 106 is an independent core module comprising the functionality of the treatment engine 109 and treatment installer 107. In other examples, the treatment configurator 106 may comprise two separate core modules—the treatment engine 109 and the treatment installer 107. In examples where the digital treatment system comprises only a single treatment package 104 the treatment engine 109 may be included as part of the treatment package 104.

The treatment instructions can also indicate patient data 115 and/or non-patient data to be acquired by the core package 102, core modules and/or treatment specific modules that offer the appropriate instrument. The indicated data may be required for processing the one or more treatment decisions. The core package 102 can acquire the patient data 115 based on the treatment instructions of the treatment descriptor 108. In this way, the treatment configuration 108 may include treatment instructions indicating the patient data 115 required to configure the one or more digital therapies 111.

An example clinical treatment decision is deciding an appropriate dose of a drug for the patient. The appropriate dose may be based on biometric data collected during previous weeks.

The treatment configuration 108 can describe the data and treatment decisions required to deliver the one or more digital therapies 111 to the patient. As a result, the treatment configuration 108 may be subject to a high degree of regulatory scrutiny.

In some examples, the treatment engine 109 may process the treatment decisions using one or more algorithms. The one or more algorithms may comprise rule-based algorithms and/or machine learning algorithms. The algorithms may be stored in memory local to the core package 102 or remote from the core package 102.

In some examples, the one or more algorithms are associated with the core package 102. For example, the one or more algorithms may be associated with the treatment configurator 106, in particular the treatment engine 109, or with one or more other core modules. In this way, the algorithms would be subject to regulatory approval when the core package 102 and/or the relevant core module(s) is subject to regulatory approval. A treatment package 104 can make use of these approved algorithms without the algorithms being subject to reapproval when the treatment package 104 is the subject of regulatory approval. Algorithms, particularly machine learning algorithms, can present a high burden for regulatory approval. By including the algorithms in the core package 102, common to a plurality of treatment packages 104, the algorithms may only require regulatory approval once or a few times. New approved treatment packages 104 can advantageously be added or updated without requiring reapproval of the core package 102, core modules or the corresponding algorithms. The treatment packages 104 may also comprise high-regulatory burden functionality, including algorithms. The treatment package 104 may include such functionality in treatment modules that can be independently assessed by the regulator. In this way, the treatment packages 104 and/or the treatment modules can be assessed independently without requiring reassessment of the core package 102 or other approved treatment packages 104.

To enable independent approval of algorithms in the treatment configurator 106, the treatment configurator 106 may comprise a safety module 113 for applying one or more safeguards to the digital therapies 111. The safety module may comprise one or more inherent safeguards and/or one or more inputs for receiving safeguards.

As an example of an inherent safeguard, the safety module 113 may impose a rule that no drug dosage can ever exceed a toxic dose for the patient. To enable this, the safety module 113 may include or access a drug database containing a list of known drugs, drug interactions, safe dosage ranges and other regulatory standards. The drug database may be updated regularly to ensure ongoing regulatory compliance. When the treatment engine 109 of the treatment configurator 106 processes a treatment decision based on a treatment instruction from a treatment configuration 108, the safety module 113 can cross check any calculated drug dosages that may form part of the digital therapy 111 against the drug database. In this way, the safety module 113 can ensure that any calculated drug dosages are within recommended safety limits. The safety module 113 may cross-check the calculated dosage against a specific entry in the drug database based on the drug and one or more parameters of the patient data 115 (for example, age, sex, ethnicity, co-morbidities, other medications etc.).

The safety module 113 may also comprise inputs for receiving safeguards from other components. For example, the safety module 113 may receive specific treatment safeguards from the treatment configuration 108 (for example from the treatment descriptor or the application configuration) or from a treatment module. In this way, the treatment configuration 108 can declare one or more safeguards for the treatment configurator 106 to enforce. The safety module 113 of the treatment configurator 106 can interpret the safeguards in the treatment configuration 108 and enforce the safeguards on the digital therapy 111. The treatment configuration 108 can extend the inherent safeguards of the safety module 113 to account for safety scenarios that may only exist in the context of the particular treatment. For example, a treatment package may include a prescribed drug and include safeguards around doses of other drugs that are known to cause adverse events when combined with the prescribed drug.

The safety module 113 may apply the one or more safeguards to limit or disable the functionality of a particular treatment (or treatments).

In one or more examples, one or more modules may include safeguards in their contract specification. For example, the safety module 113, the treatment configurator 106, the treatment engine 109 or one or more treatments specific modules may comprise a module contract specification including one or more safeguards.

In some examples, one or more algorithms for processing treatment decisions may be associated with a treatment package 104, for example in a treatment module. In this way, specific treatment algorithms can be provided to the treatment configurator 106 for processing. In some examples, the treatment engine 109 may execute the treatment decisions. In other examples, the relevant treatment module may execute the code and communicate the relevant data with the treatment configurator 106. The specific treatment algorithms can be subject to regulatory approval with the treatment package 104 and/or treatment module. Such an approach can enable new algorithms to be provided to the treatment configurator 106 while maintaining the regulatory independence of the core package 102, the core modules, the treatment package 104 and the treatment modules. In such examples, the safety module 113 would operate as described above for algorithms associated with the treatment configurator 106.

Each of the one or more treatment packages 104 may be suitable for treating a respective disease or condition. The treatment configuration 108 may provide treatment instructions to the treatment configurator 106 related to treatment decisions and/or acquisition of patient data 115 for configuring one or more digital therapies 111 for treating the disease or condition.

The disease or condition may be any of: pre-diabetes; diabetes; cardiovascular disease; neurodegeneration diseases, such as Mild Cognitive Impairment (MCI), Alzheimer's disease and Parkinson's disease; atrial fibrillation; attention deficit hyperactivity disorder (ADHD); autoimmune diseases, such as ulcerative colitis, lupus erythematosus, Crohn's disease, coeliac disease, Hashimoto's thyroiditis, bipolar disorder; cerebral palsy such as dyskinetic and athetoid; chronic graft-versus-host disease; hepatitis; chronic kidney disease; arthritis and chronic osteoarticular diseases, such as osteoarthritis and rheumatoid arthritis; cancer; obesity; asthma; sinusitis; cystic fibrosis; tuberculosis; chronic obstructive airways disease, bronchitis; bronchiolitis; pulmonary fibrosis; pain, including chronic pain syndromes; depression; eating disorders; polycystic ovary syndrome; epilepsy; fibromyalgia; viral diseases, such as HIV/AIDS; Huntington's disease; hypotension; hypertension; allergic rhinitis; multiple sclerosis; fatigue states, including chronic fatigue syndrome; insomnia; narcolepsy; osteoporosis; periodontal disease; postural orthostatic tachycardia syndrome; sickle cell anaemia and other haemoglobin disorders; sleep apnoea; thyroid disease; reflux, including gastroesophageal reflux; vomiting; irritable bowel syndrome (IBS); inflammatory bowel disease (IBD); peptic ulcer; acute urticarial; atopic dermatitis; contact dermatitis; seborrheic dermatitis; headache, including migraine, cluster headache, and tension-type headache; addiction, such as drug addiction, in particular opiate dependency, cocaine, alcohol, or nicotine addiction and chronic usage thereof; thromboembolic disease; hair loss; hormone replacement therapy; psychiatric disorders, such as psychosis, anxiety and depression; endocrine dysfunctions, including growth hormone deficiency, hypothyroidism; haematological disorders, including clotting factor 5 deficiencies or low levels of white or red blood cells; neurodevelopmental delay (NDD) disorders, including Autistic Spectrum Disorder (ASD), Smith Magenis Syndrome and ADHD; parasomnias, including REM and NREM parasomnias and nightmare disorders; sleep movement disorders, such as restless legs syndrome and periodic limb movement disorder, circadian rhythm disorders (including such disorders brought on by shift work and/or jet lag); chorea and tic disorders.

FIG. 2 illustrates a detailed schematic of a digital treatment system 200 according to an implementation of the present disclosure. Features of FIG. 2 present in FIG. 1 have been given corresponding numbers in the 200 series and will not necessarily be described again here.

System Architecture

The digital treatment system 200 comprises software processes running in one or more mobile applications 210 on a mobile platform 201 and as backend services on a cloud platform 203. The system 200 employs the modular architecture discussed above in relation to FIG. 3.

In this example, the core package 202 comprises a mobile application, or mobile app, 210. The mobile app 210 comprises a plurality of core modules of the core package 202 in the form of core client modules 246. The core package 202 further comprises core backend modules 212. In some examples, the core backend modules 212 may be included in the mobile app 210.

The mobile app 210 may be made available to a patient and installed through an official delivery channel such as an app store. The mobile app 210 may be installed on a range of local personal devices such as a personal computer, a mobile communications device, such as a tablet, a smartphone, a smartwatch or other devices known in the art.

The mobile app 210 can host the one or more treatment packages 204. The mobile app 210 may act as the primary user interface for treatment delivery. As explained above, the core package 202 can provide the core modules 246 as shared capabilities to each of the treatment packages 204. These shared capabilities can include execution of treatment configurations, data management, user account management, prescription checking and third-party device integration.

In some examples, the treatment packages 204 may be included (and integrated) with the mobile app 210 when it is installed by the patient. Access to individual treatment packages 204 may then be unlocked accordingly. In other examples, the treatment packages 204 may be downloaded separately and integrated with the mobile app 210 or installed as separate mobile applications in the mobile platform 201. Therefore, the digital treatment system 200 may provide one or more treatments to a user ranging from a mobile app 210 dedicated to one treatment that cannot be modified, to a treatment store arrangement in which a user or clinician can select or unlock a range of treatments via the mobile app 210.

The backend modules hosted on the cloud platform 203 may comprise backend services. In this example, the core package 202 and each treatment package 204 have their own respective backend services (core backend modules 212 and treatment specific backend modules 214). The backend services hosted on the cloud platform 203 may also comprise supporting systems 216. The backend services can provide supporting infrastructure for treatments, such as long-term data storage or operational monitoring. The mobile app 210 may communicate with the backend server via a communication network such as the internet or a cellular network. The backend services may constitute the “server” part of a client-server architecture arrangement.

The core backend modules 212 may comprise web services that provide shared capabilities to the digital treatment system 200. The core backend modules 212 may comprise common modules that can be called by web service clients running in the core client modules 246 of the core package 202 or one or more treatment modules 226 of the treatment package 204. The core backend modules 212 can provide server-based capabilities that apply across all treatments, such as data management, and integration with wider systems.

The treatment packages 204 can each include treatment specific backend modules 214 for aspects of the digital therapy provided by the treatment package 204 that may need to run on a backend server. Examples of treatment specific backed services 214 include video content (hosted and accessed via a cloud service), or services that can be used to query machine-learned models that are too computationally intensive to run on the local personal device.

The supporting systems 216 can comprise a suite of supplementary functionality to the digital treatment system 200. A person skilled in the art will appreciate that the digital treatment system may include any, all or none of the modules or components of the supporting systems.

The core modules 246 and treatment modules may interface with any of the supporting systems 216 through the core platform and a systems integration module 266 which is a core backend module 266. Further detail on each of the supporting systems 216 is provided below.

Treatment Package Installation

Installation of the treatment Package 204 may be static or dynamic. That is, the treatment package 204 may be installed at build time to create a complete self-contained executable binary, or at runtime, where the treatment package 204 is downloaded and dynamically integrated into the mobile application 210.

As described above in relation to FIG. 1, each treatment package 204 comprises a treatment configuration 208, which may include an application configuration 218 and a treatment descriptor 219.

The application configuration directives (app config) 218 can describe how the mobile application 210 or core package 202 is configured to support the treatment or one or more digital therapies. Installation of the app config 218 may occur during installation of the treatment package 204. The core package 202 may read the app config 218 from the treatment package 204 and register the app config 218 with one or more core modules 246 of the core platform 202. At runtime, the one or more core modules 246 can read the app config 218 and update their state and behaviour accordingly.

The app config 218 may include:
An indication of the core modules (and any treatment modules) required to deliver the treatment and whether the modules provide any medical-device functions or non-medical-device functions; and/or
Directives to define module behaviour, appearance, and personalisation (eg configuring a type of notification that should be generated each day).

For example, the app config 218 may configure a notification module 228 of the core package 202 to generate a daily notification for the patient to take medication (in this way the notification module is a core medical module). The app config 218 may allow the patient to select a specific time of notification from a predefined range of values. In a further example, the app config 218 may determine the availability of a feedback form depending on the context in which the digital therapeutic is provided.

As described above, the treatment descriptor 219 can include the treatment instructions and an indication of data required for the respective treatment package 204. The treatment descriptor 219 crosses the interface 217 from the treatment package 204 to the core package 202 by: i) being registered as a treatment that must be run by the treatment engine module 209; and ii) being digitally read by the treatment engine 209 at run time. The treatment engine 209 can interpret the instructions in the treatment descriptor 219 and deliver the treatment.

A treatment package 204 may also comprise one or more custom treatment modules 226. A treatment module 226 may comprise medical device compliant software developed for meeting the intended use of the treatment package 204. The one or more treatment modules 226 may be installed through static linking (for build time installation of the treatment package 204) or dynamic linking (for runtime installation of the treatment package 204). In this way, the core platform of the core package 202 (or another treatment specific module 226) may call the one or more treatment modules 226 as required. Instructions describing when components of the core package 202, such as the treatment engine 209, should call a custom treatment module 226 may be provided or indicated by the application configuration 218 or the treatment descriptor 219.

One example custom module 226 is a machine-learned model and supporting algorithm capable of classifying patients according to their response to a set of questions.

Each treatment module 226 may define one or more requirements. The core package 202 (for example the core platform) may ensure these requirements are met during installation of the treatment package 204 or during subsequent processing of the treatment package 204. The one or more requirements may include:
required minimum platform version;
required hardware version and/or features; and
any additional system configuration required by the module.

Additionally, each exported instrument that may be output by the treatment module 226 may include information based on the module performance requirements. For example, the exported instrument may include one or more of:
An instrument description;
An identifier of the exported instrument (In some examples, different modules can export instruments with the same identifier, to allow an instrument lookup and matching);
Data provided by the exported instrument, a specification of behaviour, and other meta-data used to perform instrument certification; and
Representative use cases (ie test specifications and automated test cases)

The treatment module 226 may impose input constraints on each imported instrument based on the module performance requirements. The input constraints may include one or more of:
The identifier of instrument that is required;
The acceptable instrument policy that must be met; and
The specification of behaviour and data required (the contract specification)

The platform requirements, acceptable policies and contract specification of the treatment module 226 form an overall specification of required operational parameters. The core package 202 and/or treatment module can ensure compliance with the specification to enable the treatment module 226 to be safely integrated into the digital treatment system 200.

The core package 202 and/or the treatment module 226 may check the following:
the core package 202 or core platform within which the treatment module 226 runs is a valid version, and configured according to the requirements of the treatment module 226;
the hardware complies with version and feature set requirements; and
imported instruments required by the treatment module 226 are present, meet the import policy, and can be certified against the contract specification of the treatment module.

The requirements of the custom treatment modules 226 and associated instruments and the monitoring of their compliance by the core package 202 provides and enforces the modular isolation described above in relation to FIG. 3. This in turn enables the partitioning of the digital treatment system 200 allowing the treatment package and/or treatment modules to be independently updated and/or subject to regulatory approval. A person skilled in the art will appreciate that in some examples, the requirements and compliance monitoring outlined here in relation to a treatment module 226 can equally be applied to any of the core modules 246. This in turn enables the independent update and regulatory approval of any of the core modules 246 or the core platform, as indicated above in relation to table 1.

The digital treatment system 200 can personalise the digital therapy for a user during installation, and may be further personalised during treatment delivery. The personalisation is separated into clinical and non-clinical, the former being adjustment of the treatment, the latter being adjustment to encourage treatment adherence.

For example, the system 200 may provide an initial clinical personalisation by setting a starting dose for a patient based on their age and weight. The system 200 may provide an initial non-clinical personalisation by setting a tone of voice used in notification messages based on a user's response to onboarding questions.

The treatment package 204 may initiate the personalisation by providing personalisation settings via any of the following:
Directives of the app config 218 which the core package 202 can direct to one or more core modules 246 required for the treatment;
Treatment instructions of the treatment descriptor 219 which may be executed by the treatment engine 209;
Treatment modules 226 initiating their own personalisation processes; and
A specific non-treatment personalisation module 222.

Personalisation may be based on patient data acquired by the core package, for example, user input data or data from other sources, such as an electronic health record (EHR).

The non-treatment personalisation module 222 may comprise further configuration directives for personalising the delivery of the treatment for the patient (as opposed to personalisation of the treatment itself). These directives are a type of application configuration focussed on optimising non-clinical elements of treatment, such as improving patient adherence to a treatment regimen. In some examples, the personalisation settings 222 may comprise a set of default personalisation settings 222 when the treatment package is first installed. The personalisation settings 222 may be configured to adapt automatically based on patient behaviour and/or may be configured for adjustment by the patient.

An example of a non-treatment personalisation setting 222 may be suggesting or selecting new notification times based on patient behaviour or engagement. The notification module 228 may receive settings from the treatment package for personalising the notification times. The treatment package 204 may include many other personalisation settings 222 based on the capabilities of the core package 202 and/or the treatment specific modules 226.

The core package 202 may offer a standard UI architecture with core modules 246 including standard UI displays, standard branding assets 230, standard pages 232 and standard navigation mechanisms shared across the one or more treatment packages 204. For example, the UI assets 220 of the individual treatment packages 204 can augment the standard UI architecture with one or more customised: colour schemes, branding, layouts, navigation structures, form configurations, UI displays or other UI assets.

The UI assets 220 may be integrated into the mobile application 210 during treatment package installation. The core package 202 can then read the UI assets 220 at runtime to render the UI. An example of a custom UI asset 220 is a visual widget for measuring the patient's visual response to collect a treatment specific biometric related to their cognitive function. The custom visual widget would form part of a treatment package 204.

In some cases, integration of a new screen may require lower-level integration, for example with a third party User Interface framework used by the platform.

A treatment package 204 may include media content 224 for delivery prior to, during or subsequent to a digital therapy. The media content 224 may comprise rich text, audio, video, or other typical media. The media content 224 may be clinically significant, e.g. training videos for using biometric devices, digital CBT lessons, or patient information sheets.

The treatment package 204 may store the media content 224 locally on the mobile platform 201 or remotely on the cloud platform 203 as a treatment backend service 214. During installation of the treatment package 204, the media content 224 may be extracted onto either local storage or networked storage accessible to the core package 202. Alternatively, or in addition, the networked location of any remotely stored media content 224 may be registered with a suitable core module 246 (e.g. a content manager 234) in the core package 202. The media content 224 can then be retrieved or streamed when accessed by a patient.

The core package 202 may make the media content 224 available to the patient using mechanisms such as lesson lists or prompts to view content during patient onboarding.

Core Modules 246, 212

In this example, the core package 202 comprises a core platform (not illustrated) and the following core modules 246 operating on the core platform:

Core Client Modules 246
An application framework 250 may comprise a standard application user interface for hosting treatments. The application framework may provide application navigation, splash screen, and similar application features shared across all treatment packages.
A standard pages module 232 may comprise standard UI screens shared across treatment packages 204, for example a treatment dashboard screen showing available treatments, and a task screen showing a consolidated view of current tasks for the user to perform.
A branding assets module 230 may comprise digital assets for displaying to the user. A default set of assets may be provided. Specific treatment packages 204 may override some or all of the branding assets (or other, similar UI assets that make up the theme of the application).
A content manager module 234 may be responsible for providing access to content required as part of a treatment, as described above in relation to the media content of treatment package 204. The content may be local to the core package 202, form part of the media content 224 of the treatment package 204 and/or be accessed via a backend service 212, 214.
A feature manager module 252 can enable or disable application features. The core package 202 may use the feature manager module 252 to allow a treatment to be delivered in either trial or real-world settings. The feature manager module 252 may also enable live adjustment of application features in an A/B setting (if adjustment is acceptable in the context, eg to account for randomisation in a clinical trial).
The treatment installer 207 may form part of the treatment configurator 206 as discussed above in relation to FIG. 1. The treatment installer 207 may be responsible for downloading and installing treatment packages 204. The treatment installer may also detect required treatment updates and may lock treatments if they have been withdrawn.
The treatment engine 209 may form part of the treatment configurator 206 as discussed above in relation to FIG. 1. The treatment engine 209 may provide an execution environment and engine for the treatment packages 204, including the treatment descriptor 219 and treatment modules 226. The treatment engine 209 may take the form of a virtual machine sandbox environment for executing treatment processes.
An authentication module 254 may integrate the mobile application 210 with standard authentication providers. This may include user interface elements as well as third party API integrations. The authentication module 254 may communicate with the user profile module 268 in the supporting systems via a systems integration module 266.
A prescription checker module 256 can manage user access to treatment packages 204. The prescription checker module 256 can ensure that a user has a relevant prescription or has purchased the relevant treatment over the counter (OTC) before allowing access to a treatment packages 204. The prescription checker module 256 may communicate with the prescription management module 272 in the supporting systems 216 via the systems integration module.
The notification module 228 can manage aspects of the mobile application 210 related to notifications, such as registering the need for specific notifications, enabling/disabling notifications, or aggregating notifications to avoid overloading the user. The notification module may provide medical device functions by instructing the patient to perform a therapeutic task.
A treatment data store module 236 can store treatment data or patient data on the client device, including any of: user-input data, readings from a connected device and data retrieved from backend services such as electronic health records. The treatment data store module 236 may also manage access to the stored data for the core package 202, the core platform, other core modules 246, the treatment packages 204 or treatment modules. The treatment data store module 236 may also integrate with a treatment data replica module 262 for backup/recovery purposes. The treatment data store module 236 may also enforce information governance policies.
A device integration module 258 can integrate the digital treatment system with connected devices. The device integration module 258 may receive data from connected devices for use in a treatment, for example in processing a treatment decision. In some examples, the device integration module may instruct a connected device to deliver a therapy as part of the treatment.
An error handler module 260 may record error information and transmit the error information to backend core modules 212, such as the treatment watchdog module 264, or to supporting systems 216, such as the patient support module 276, for diagnosis and support.

Backend Core Modules 214
The treatment data replica module 262 can comprise a service for replicating data captured in the mobile application 210, ensuring it is available for backup and recovery purposes (to ensure treatment continuity) and clinical use if appropriate.
The treatment watchdog module 264 comprises a server-side module for detecting problems in treatments, such as non-conformance or failure to transmit data for backup.
A system integration module 266 can provide a set of server-side integrations with the supporting systems 216.

Supporting Systems 216

The supporting systems 216 can comprise a suite of supplementary functionality to the digital treatment system 200. A person skilled in the art will appreciate that the digital treatment system may include any, all or none of the modules or components of the supporting systems. In this example, the supporting systems 216 comprise:
A user profile module 268 which may provide user authentication services for the mobile application 210. The user profile module may enable registration, authentication, profile retrieval, and other common user profile management capabilities.
A patient data module 270 comprising a backend service and user interface for managing access to patient data stored in an electronic health record system. The EHR may be provided externally or form part of a patient health record module 274. The patient data module 270 may control access to the patient data based on information governance policies.
The prescription management module 272 can manage user access to treatment packages. The prescription management module 272 may integrate with external provider systems provided by healthcare intermediaries and use the resulting information to provide access control information to the mobile application 210.
The patient record module 274 comprising a data repository of patient health records relating to a user. The health records may include those generated by the mobile application 210 as well as data retrieved from third party EHR systems. Records in the repository may be retrieved for use by the mobile application 210 for the treatments, shown to clinicians (in a controlled manner, such as via the patient data module 270), or submitted for storage in third party systems.
A patient support module 276 may provide support to patients during treatment, for example by reporting treatment status, adverse events, or other similar clinical information. The patient support module may be used by clinical staff to provide treatment support.
An analytics module 278 can capture patient application usage data to support product development. The captured data may be anonymised, and may include time spent on application screens, user actions, and similar data.
A treatment operations module 280 can enable remote access for operational support to perform routine operational activities related to patient treatments, such as handling technical issues or non-clinical application usage problems.
A clinical trial data store module 282 may provide a system for storing data captured during clinical trials.

Treatment Delivery

Following installation of a treatment package 204, the core package 202 can deliver the respective treatment. The treatment package 204 and the core package 202 can interact to deliver the treatment.

Data Capture

The core platform may configure one or more of the core modules 264 to capture data. In some examples, the core platform may receive instructions to configure the core modules 264 via the application configuration 219 (in the treatment package 204). In other examples, the core platform may receive instructions from the treatment engine 209 as it determines that particular data is required to execute the treatment descriptor 219. The treatment descriptor 219 itself may therefore indicate the data required to deliver the respective treatment.

As described above data may be captured using screen-based user input, for example, a daily medication log, from EHRs, for example for patient height or weight, or from an attached (medical) device such as a blood pressure monitor, among other potential sources.

In some examples in which the core package 202 is hosting multiple treatment packages 204, the core package 202 may consolidate the data capture requirements of the multiple treatment packages 204. For example, a core module, such as the treatment configurator 206 or the notification module 228, or the core platform may determine that two treatment packages 202 require the same patient data, such as a blood pressure measurement. In response, the core platform may instruct the device integration module 258 to request a single data input. The core platform may then communicate the resulting data input to the treatment configurator 206 or relevant treatment modules 226 for use in both treatment packages 204.

Treatment Decisions

In some examples, the treatment engine 209 may execute the one or more treatment decisions by interpreting or otherwise executing the treatment descriptor 219. The treatment descriptor can include the treatment instructions describing clinically significant, safety critical treatment decisions required to deliver the respective treatment. In other examples, the treatment engine 209 may be omitted or not used. One or more core medical modules or treatment modules may also execute a treatment decision.

The treatment engine 209 can receive the treatment descriptor 219 and determine any data required to process the treatment decisions. The treatment engine 209 may communicate with one or more core modules 246 (for example the device integration module 258) (and/or treatment modules 226) to obtain the required data. As outlined above, the app config 218 may also configure the core modules 246 (and/or treatment modules 226) to acquire data. The treatment engine 209 can then receive the acquired data and execute the treatment decisions, determine one or more clinical conclusions and provide corresponding results.

The treatment engine 209 may process the treatment decisions and provide results at a particular time or interval of time, for example once a day. The treatment engine 209 may also process the treatment decisions and provide results in response to one or more activities or events, for example, an unexpected change in a biometric reading such as blood pressure.

Personalisation (Ongoing)

As outlined above, the digital treatment system 200 may perform ongoing personalisation for the treatment and application experience of a user. The system 200 may adapt the personalisation in response to newly gathered data. Any component of the system 200 may be responsible for personalisation, for example, a treatment package 204 may perform, and/or provide instructions for the core package 202 to perform the personalisation. As an example, the system 200 may reduce a dose of a drug in response to a patient reported symptom severity. In another example the notification module may modify a tone of voice used in treatment messages may be modified based on observed adherence levels. Treatment personalisation may be initiated according to instructions in the treatment descriptor 219 or by core modules or treatment modules that deliver the treatment or patient experience. In some examples, the core package may provide continuity of user personalisation from a first treatment package to a subsequent treatment package.

Interactions Between Treatments

The core package 202 may host multiple treatment packages 204 concurrently and thereby can offer multiple treatments to a patient at the same time. A treatment configuration 206, for example, the treatment descriptor 219, may include interaction directives designed to account for treatment interactions. The treatment interactions may include drug interactions and the interaction directives may indicate lower dosages of a first drug if a patient is taking certain other drugs. The treatment configurator 206 or the treatment engine 209 may read the interaction directives from the respective treatment configurations 208 of the treatment packages 204 and determine one or more treatment interactions. The treatment configurator 206 may then set guardrails in the safety module (see FIG. 1) based on the interaction directives. For example, the guardrails may ensure that appropriate lower doses are indicated to the patient for a drug interaction.

In some examples, the core package 202 may run different instances of the treatment configurator 206, one for each treatment package 204. Each instance of the treatment configurator 206 may read and execute the treatment configuration 208 from a respective treatment package 204. The multiple instances of the treatment configurator 206 may then communicate with each other to exchange and determine the interaction directives.

Treatment Lifecycle Management

The digital treatment system 200 can manage the installed treatment packages 204 on an ongoing basis. For example, the digital treatment system 200 may update, lock or withdraw a treatment.

Updates

New versions of treatment packages may be released, as a result of (for example) new clinical evidence, user interface enhancements, new product features, and/or changes to capabilities of the shared core package 202.

Independent update and regulatory approval of different elements of the digital treatment system 200 is an advantage of the disclosed partitioned system. The system 200 may update the various components and modules (see FIG. 3 and Table 1) independently of one another as follows:
Treatment Packages 204—As an example, the digital treatment system 200 may download an updated treatment package with a modified treatment configuration 206 or a new version of a treatment-Specific module 226 with improved performance.
Core (common) Modules 246—As an example, the digital treatment system 200 may download a new version of a core module that is used for several treatment packages or download a completely new core module introducing new functionality. The core modules 246 may be core medical modules or core non-medical modules.
Platform and System layers—The digital treatment system 200 may update the platform layer or the system layer to provide new or extended capabilities. The two layers may be updated together or separately.

The digital treatment system 200 may only process an update of a component following one or more operational checks to ensure to ensure the safety and integrity of any treatments being delivered are maintained. The system 200 may perform one or more of the following operation checks:
Version compatibility checks between treatment Packages 204, modules (core and treatment) and other software elements (platform and system layers).
Imported Instruments meet the certification requirements and acceptable instrument policies. For example, a new version of a module may no longer meet the acceptable instrument policy of a second module that uses it. In such instances, the system 200 may reject the update or suspend the relevant treatment package 204.
System-level constraints on the composition of modules and platform are met, to ensure the overall composition is acceptable from a risk and regulatory perspective. Such constraints may include checking that the composition has been pre-approved from a risk management perspective, or that the memory footprint of the combination of modules is within tolerances for each specific module.

It will be appreciated that one or more core modules (for example the treatment installer 207, the feature manager 252), the platform layer and/or the system layer may perform the update and operational checks.

Treatment Locking/Withdrawal

In some examples, the digital treatment system 200 may withdraw a treatment package, for example, due to a safety concern. The core package 202 may interact with the relevant backend service, for example the treatment watchdog module 264, or the patient support module 276 to detect the withdrawal of a treatment package 204. The system can then lock access to the treatment, as required.

Patient Onboarding

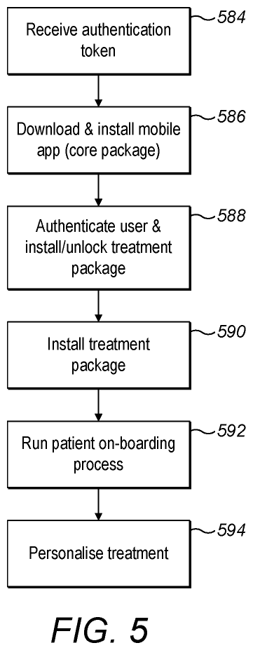

FIG. 5 illustrates an example end-to-end process for user interaction with the digital treatment system according to an implementation of the present disclosure. The process commences with a first encounter with a healthcare professional, through prescription, download, installation, and personalisation of the digital treatment.

In a first step 584, a user receives an authentication token for accessing the digital treatment system and/or one or more treatment packages. For example, a user may be prescribed a drug/digital combination by a clinician who can explain to the patient that there is a digital component of their treatment comprising the digital treatment system. The pharmaceutical may be dispensed per existing pathways (in person at a pharmacy, or via delivery if a repeat prescription) and provide an authentication token such as a QR code, a product code on the packaging or information leaflet. In other purely digital examples, the user may receive the authentication code directly from the clinician. The clinician may create a user profile in the user profile module on behalf of the user.

In a second step 586, the user downloads the mobile application. The mobile application may be downloaded from an application store or internet link as is known in the art. The authentication token may comprise a download link. For example, a user may scan a QR code with a camera of a mobile device which may commence the download.

In a third step 588, the digital treatment system authenticates a user and installs or unlocks one or more authorised treatment packages. For example, the system may instruct a user to input their authentication token. The system may then download, install and/or unlock one or more treatment packages as authorised by their authentication token.

In some examples, the authentication token may be a generalised code enabling access to treatment package x. In some examples, the authentication token may enable access to treatment package x in association with drug y. That is the authentication token may encode details of the contents of a drug packet. The authentication may include patient data including name, address, and instructions on dosage. As a result the authentication token may enable access to treatment package x in association with drug y for patient z and prescription alpha. The authentication token may further provide details of the user that enables the system to access EHRs and other patient data. Access to such data may also be established when a clinician creates a user profile. The authentication token may register and authenticate the mobile device or mobile application instance with the user profile in the user profile module 268. In some examples, the authentication token may be single-use to prevent abuse.

In a fourth optional step 590, for examples in which the system installs the treatment package, the treatment configuration indicates any core modules required and the core package registers these modules with the respective treatment.

In a fifth step 592, the system runs an on-boarding process with the user. Data required for the treatment may be collected directly from the user. Patient data may also be retrieved from an EHR, if available. The system may access the EHR via the user profile module, the patient data module and/or the patient record module.

In a sixth step 594, the system may personalise the treatment to the user. The system may perform an initial personalisation and/or an ongoing optimisation based on received data and/or data generated through the interaction of the user with the application. As outlined previously, treatment personalisation may be performed as a result of instructions in the treatment descriptor, through directives in the application configuration which can modify module behaviour, and/or through treatment module specific processes which can be initiated independently. Patient data may influence the personalisation.

1. A digital treatment system, comprising:
a core package; and
a treatment package;
wherein the core package is software partitioned from the treatment package,
wherein the core package is adapted to:
receive a treatment configuration from the treatment package; and,
configure one or more patient treatment therapies based on the treatment configuration.

2. The digital treatment system of claim 1, comprising a plurality of software modules, the plurality of software modules comprising:
at least one core module in the core package; or
at least one treatment module in the treatment package; or
at least one core module in the core package and at least one treatment module in the treatment package.

3. The digital treatment system of claim 2, comprising a plurality of core software modules, wherein the core package comprises a core platform and the plurality of core software modules are configured to operate on the core platform.

4. The digital treatment system of claim 2, wherein each of the plurality of software modules is a separated partition of software with one or more associated module performance properties.

5. The digital treatment system of claim 4, wherein the one or more associated module performance properties include one or more of:
module functionality provided by a first software module of the plurality of software modules;
module performance constraints required by at least one of the first software module or one or more other software modules of the plurality of software modules;
inbound information constraints and outbound information constraints on data or information being passed to or from the software module; and
descriptive properties for enabling at least one of the one or more other software modules or the digital treatment system to determine compatibility of the first software module with performance constraints of the respective other software module.

6. The digital treatment system of claim 2, wherein:
each of the plurality of software modules is configured to register a module interface with the core platform indicating at least one of:
exported instruments comprising services or data that the software module can provide to other parts of the digital treatment system.

7. The digital treatment system of claim 6, wherein the core platform is configured to match an instrument request from a first software module of the plurality of software modules with one or more exported instruments indicated by the registered module interface of a second module of the plurality of software modules.

8. The digital treatment system of claim 7, wherein:
the first module is configured to provide an instrument request to the core platform with an associated contract specification indicating one or more constraints on the matched instrument; and
the core platform is configured to match the instrument request from the first module based on the contract specification.

9. The digital treatment system of claim 7, wherein the core platform is further configured to provide a reference interface to the first module for communication with the second module.

10. The digital treatment system of claim 9, wherein the reference interface comprises:
a direct interface between the first module and the second module; or
an indirect interface comprising an intermediary between the first module and the second module.

11. The digital treatment system of claim 1, wherein the core package is further adapted to receive patient data and configure the one or more patient treatment therapies based on the patient data.

12. The digital treatment system of claim 1, wherein the core package comprises a treatment configurator for receiving the treatment configuration from the treatment package and configuring the one or more patient therapies.

13. The digital treatment system of claim 1, wherein the treatment configuration comprises one or more treatment instructions comprising at least one of:
one or more treatment decisions;
an indication of one or more modules of the core package required to configure the one or more patient treatment therapies;
an indication of one or more treatment modules for registering with the core platform; and
an indication of patient data required by a treatment engine to configure the one or more patient treatment therapies.

14. The digital treatment system of claim 13, wherein the treatment configurator comprises a treatment engine configured to process the one or more treatment decisions using one or more algorithms.

15. The digital treatment system of claim 14, wherein:
at least one of the one or more algorithms is associated with the core package; or
at least one of the one or more algorithms is associated with the treatment package; or
at least one of the one or more algorithms is associated with the core package and the treatment package.

16. The digital treatment system of claim 1, wherein the treatment configurator comprises a safety module configured to apply one or more safeguards when configuring the one or more patient treatment therapies.

17. The digital treatment system of claim 16, wherein the safety module comprises one or more inherent safeguards for configuring the one or more patient treatment therapies.

18. The digital treatment system of claim 17, wherein the safety module is configured to:
access a drug database comprising drug data; and
determine one or more inherent drug safeguards based on the drug data.

19. The digital treatment system of claim 16, wherein the treatment configuration comprises one or more treatment specific safeguards for configuring the one or more patient treatment therapies.

20. The digital treatment system of claim 16, wherein the one or more safeguards are associated with a contract specification of a corresponding software module.

21. The digital treatment system of claim 1, further comprising one or more further treatment packages software partitioned from the core package, each treatment package of the one or more treatment packages comprising a respective treatment configuration and wherein the core package is adapted to receive each treatment configuration from the respective treatment package and configure one or more respective patient treatment therapies based on the treatment configuration.

22. The digital treatment system of claim 1, wherein the one or more treatment therapies comprise one or more digital therapies, and wherein the one or more digital treatment therapies comprise at least one of a pharmacological therapy comprising an indication of a dosage regime, and a non-pharmacological therapy.

23. A computer implemented method of configuring digital therapy in a digital treatment system comprising:
receiving at a core package of the digital treatment system a treatment configuration from a treatment package software partitioned from the core package; and
configuring the one or more digital therapies based on the treatment configuration.

24. The method of claim 23, wherein configuring the one or more digital therapies comprises:
registering and configuring one or more software modules with a core platform of the core package for use in the digital therapy; and
wherein each of the plurality of modules is a separated partition of software with associated module performance properties.

25. The method of claim 23, wherein configuring the one or more digital therapies further comprises:
acquiring data for use in the one or more digital therapies; and
processing one or more treatment decisions based on the data.

26. A digital treatment system, comprising:
a treatment package relating to a patient therapy;
a core package comprising a core platform, the core package adapted to configure the patient therapy; and
a plurality of software modules including at least one of:
one or more core modules in the core package; or
one or more treatment modules in the treatment package,
wherein the core platform is configured to integrate the plurality of software modules such that the core package is software partitioned from the treatment package.
